(19) United States
(12) Patent Application Publication
(10) Pub. No.: US 2018/0130555 A1
(43) Pub. Date: May 10, 2018

(54) SYSTEMS AND METHODS FOR INTELLIGENT ADMISSIONS

(71) Applicants: George Chronis, Chesterfield, MO (US); Erik E. Stone, Columbia, MO (US); Donald A. Viragh, Weldon Springs, MO (US)

(72) Inventors: George Chronis, Chesterfield, MO (US); Erik E. Stone, Columbia, MO (US); Donald A. Viragh, Weldon Springs, MO (US)

(21) Appl. No.: 15/804,631

(22) Filed: Nov. 6, 2017

Related U.S. Application Data
(60) Provisional application No. 62/417,867, filed on Nov. 4, 2016.
(60) Provisional application No. 62/417,872, filed on Nov. 4, 2016.

(51) Int. Cl. G16H 40/20; G16H 50/30; G06K 9/00

(52) U.S. Cl. CPC G16H 40/20; G06K 9/00348; G16H 50/30

(57) ABSTRACT

Systems and methods for assisting in getting additional information to a triage nurse, or other healthcare individual who needs to triage or otherwise assess a patient, quickly. This information is generally obtained by performing automatic computer assessments of patients as they arrive at the emergency admission area and may be performed without the patient's knowledge such evaluation is occurring. This analysis can then be used to both directly provide additional information to a decision maker or can be used to provide specific gateways which, if passed, provide additional information.

### CROSS REFERENCE TO RELATED APPLICATION(S)

This Application claims the benefit of U.S. Provisional Patent Application Ser. No. 62/417,867, filed Nov. 4, 2016 and U.S. Provisional Patent Application Ser. No. 62/417,872, filed Nov. 4, 2016. The entire disclosure of all the above documents is herein incorporated by reference.

### BACKGROUND OF THE INVENTION

### 1.Field of the Invention

This disclosure is related to the field of healthcare services mid systems and methods for increasing the efficiency and utilization of healthcare services and resources. More particularly, it is related to systems and methods for assisting in determining the specifies about a person entering a healthcare facility through analysis of their gait to better triage admissions to the facility.

### 2. Description of Related Art

There is a strange contradiction in emergency medicine. Specifically, many of the conditions patients go to an emergency room to treat are not actually emergencies. That is, many emergency room patients do not require immediate, or even that urgent, care to avoid further injury or death. In fact, only about 3% of emergency room patients actually require immediate treatment. Instead, many patients arrive with possible emergencies, or simply with situations which do not require immediate care, but require more emergent care than may be available elsewhere at that time. This contradiction has actually led to problems in emergency treatment as there are too many potential patients arriving for medical personal to handle all of them on an immediate basis, resulting in the need for emergency rooms to have emergency patients wait for care. This has resulted in deaths, and even discharges, of patients that should have been provided immediate care. To deal with the concern that a patient with an actual urgent need is forced to wait most emergency rooms utilize a triage nurse and triage systems to attempt to quickly prioritize admissions and responses to those with the most urgent conditions.

One manner of triaging at this stage is known as the Emergency Severity index or “ESI”. The ESI provides for questions and determination points for classifying new patients into five categories based on the severity of the emergency that brings them to the admissions area as well as a determination of how long a patient can safely wait for care. ESI-1 classification is the identification of a true emergency where immediate care is required while ESI-5 is a situation of no emergency. While the ESI has unquestionably assisted in triaging in busy emergency rooms, it has some issues. The biggest of these is that ESI classification can be something of a guessing game. Patients arrive at the emergency room or urgent care center complaining of various symptoms, and a triage nurse must quickly assess each patient and triage them into an ESI category as quickly as possible. They may also need to begin treatment for some patients. However, the severity of a given person's ailments may not necessarily manifest as readily apparent distress. While acute trauma, such as a bleeding wound, is easy to triage via visual inspection alone, complications from chronic conditions-or internal injuries may be more subtle.

This need to quickly triage can mean that serious, life-threatening conditions, such as cardiac distress or stroke, which may not necessarily manifest in a visibly perceptible manner, may be given lower priority than the severity of the condition merits. Similarly, a low risk condition, which simply is manifesting as something more concerning, can also lead to a higher priority than is warranted. Either of these situations can later lead to loss of life or increased injury if the correct patient is not given the more urgent attention. Basically, triage needs to get each classification right as both an assessment which is too high, and one which is too low, being threating to a patient (whether the one being assessed, or another, forced to lower priority).

One of the problems with triage and the ESI is that the determination is often made on very-little information and, as the ESI itself even acknowledges, is often based on the professional “sixth sense” of a skilled triage nurse. One of the major problems with the lack of information is that an individual generally arrives at an emergency care facility as a John or Jane Doe. There are two primary reasons for this. The first is that emergency care is often provided as part of a medical facility that an individual does not go to for non-emergent care and the second is that identification has traditionally required an interaction and time to carry out.

On the first issue, a patient's prior medical history can be one of the most valuable indicators in the severity of their current situation and this may not be immediately known upon admission as the patient may be at a facility they have not previously been admitted to and, even if they are a prior patient, locating their records may take unnecessary time due to the need to determine their identity as part of the triage process. The very nature of emergency care will generally mean that it is being sought outside of the standard care sought. Because of this, immediate identifier (such as bracelets to identify major medical conditions such as drug allergies) which travel with an individual are very common.

The second problem with the anonymity of arrival is that the identification of the patient requires contact with the triage nurse. Thus, for very busy emergency rooms, even if all the information on the patient is readily available, it still may take time for information to get to the nurse. This problem can be additionally problematic due to language or communication barriers particularly where a patient may be in pain or have a situation where their communication is hampered. In effect, even if all the records are available, there is still a need to have a human interaction to get the records and background and such interaction are limited by available personnel and individual skill.

The issue of the need to obtain medical history on a patient is particularly problematic in light of additional concerns over the security of patient medical data. HIPAA and other related legislation has made it so that various types of medical data are not readily accessible, particularly outside of a care facility where the patient received the care in the records. Maintaining privacy can also often slow the admissions process because care must be taken not only related to the speed of obtaining the information, but of the method in which it is obtained. Further, privacy can present additional concerns in transferring information between facilities which, as indicated above, is a major issue for emergency facilities precisely because they are not the normal point of care.

Because of all these problems related to the initial anonymity of the patient, initial determinations are often based on elements perceived by a nurse and, indeed, the nurse's skill in correctly identifying conditions and classifying patients. However, what forms this skill is often exposure to the admissions area and simply experience. The longer a triage nurse is at their post, the easier it is for them to identify potential conditions and to remember questions and tests that can be the most useful in quickly sorting through them. Further, there is little doubt that additional information about a patient can assist any skilled professional, such as the triage nurse, in correctly reacting.

In addition to admission, both the provision of treatment and on the other side of treatment, discharges, have similar concerns. When a patient appears to be getting well, and shows no obvious physical signs of discomfort or distress, hospital staff may be tempted to discharge the patient in order to empty a room or bed, and reduce the overall cost of the healthcare services. The patient, likewise, is typically eager to leave, for the same reasons. However, a patient discharged too soon, may experience complications, such as dizziness or a fall, which can place the patient right back in the hospital. These circumstances can disallow the beneficial cost effects of an early discharge, and place an additional, avoidable drain on limited healthcare resources, driving up costs. Additionally, the patient suffers further injury and complications, resulting in time away from family and work. Again, a method of alerting hospital staff that a patient, who otherwise appears well, should not yet be discharged, is needed.

### SUMMARY OF THE INVENTION

Described herein, among other things, are systems and methods that collect and aggregate real-time patient data to assist in categorizing individuals according to the urgency of needed treatment. This risk may pertain to falling, or other, similar risks. The systems and methods may also issue warnings before the risk occurs, or detect the risk condition as it occurs, and alert against such conditions. The systems and methods provide monitoring, assessment, and alerts to enable healthcare professionals to proactively intervene, and potentially prevent, adverse health events.

Specifically, the systems and methods discussed herein will generally assist in getting additional information to a triage nurse, or other healthcare individual who needs to triage or otherwise assess a patient, quickly. This information is generally obtained by performing automatic computer assessments of patients as they arrive at the emergency admission area and may be performed without the patient's knowledge such evaluation is occurring. This analysis can then be used to both directly provide additional information to a decision maker or can be used to provide specific gateways which, if passed, provide additional information.

There are described herein, among other things, a system and a method for assisting in determining the urgency of admission in an admitting room of a care facility, the system comprising: a plurality of depth cameras, said plurality imaging an individual walking into said admitting room to determine said individual's gait; a computer, said computer taking in said imaging and comparing said individual's gait against stored gaits; if said individual's gait matches a specific gait of a prior patient at said care facility, prioritizing admission of said individual based on an expected priority of said prior patient; and if said individual's gate does not match stored gaits, providing said individual normal admission to said care facility.

### BRIEF DESCRIPTION OF THE DRAWINGS

FIG. 1 depicts a system for implementing the intelligent admissions systems and methods described herein.

FIG. 2 depicts an embodiment of a method for the first analysis of an intelligent admissions system and method related to detection of information indicating that the patient is at a heightened risk of severe emergency and should have prioritized triage and/or admission.

FIG. 3 depicts an embodiment of a method for the second analysis of an intelligent admissions system and method related to identifying a patient to provide secure access to correct medical records.

### DESCRIPTION OF THE PREFERRED EMBODIMENTS

An individual's particular manner of walking, sometimes referred to as their gait and meaning the articulation of the limbs and torso during a walking motion, getting up to commence a walking motion, or other elements of body movement related to ambulatory movement, is emerging as an indispensable tool in the diagnosis of frailty and fall risk, particularly among the elderly. The present disclosure is directed to the use of the relationship between the gait of an individual, and the ability to correctly and quickly obtain information about them upon their arrival at a care facility, often before they interact with a triage nurse, to be used to help make the triaging accurate.

The triage determination assistance provided by the systems and methods discussed herein comes in two primary forms. The first is using gait to detect emerging, urgent health conditions of that individual, in particular pertaining to fall risk which can identify potentially major concerns such as cardiac events as well as preventing secondary injuries. This will often be used to automatically upgrade a patient at arrival to an emergency room or other medical facility to a higher priority for evaluation. Often, this will be used to indicate to a triage nurse that this patient, should be evaluated before other patients regardless of time of arrival. The second form is to use particulars of an individual's gait as a way to accurately and correctly identify them to allow for the transfer of prior health information to a care facility in a secure manner preserving privacy and allowing a triage nurse to have very quick access to prior records, regardless of where they are stored. This method will often provide for a gatekeeper function where a likely medical record of the patient is obtained, but is not provided to the triage nurse, until the patient's identity can be secondarily confirmed or the situation is deemed sufficiently emergent to warrant provision.

The way that a person walks, including subtleties not detectable to the unaided human eye, can provide a predictor or indicator of an imminent health event and anonymous identification of an individual. The present disclosure discusses embodiments of systems and methods which allow for detection of patterns within an individual's gait to better identity urgent health conditions quickly, and the use of such detection to provide for improved efficiency of patient admission and discharge.

Many of the systems and methods described herein generally include the use of computer software to collect and examine data concerning potential patients both prior to admission and during such patient's stay in order to estimate the urgency of either that patient's condition, or another patient's condition, for admissions, and to estimate discharge dates and to obtain additional external information, about a patient without concerns over confidentiality breaches. Instead, the systems and methods use factors such as, but not necessarily limited to, bed occupancy, patient weight, patient mass, patient walks, patient height, and gait data extracted or developed automatically using sensors, or sensor systems, to determine if the individual is at an increased likelihood of having a health condition requiring immediate intervention.

While a variety of techniques and systems can be used for data gathering to feed the computer systems and methods of the present invention, there are a variety of systems and methods of data gathering which are used in certain preferred embodiments. Specifically, in an embodiment of the present invention, gait may be analyzed using the integrated sensor network described in U.S. Utility patent application Ser. No. 12/791,628, filed Jun. 1, 2010, the entire disclosure of which is incorporated herein by reference. The systems may also or alternatively include, but are not necessarily limited to, the anonymized video analysis methods and systems described in U.S. Utility patent application Ser. No. 12/791,496, filed Jun. 1, 2010, the entire disclosure of which is incorporated herein by reference.

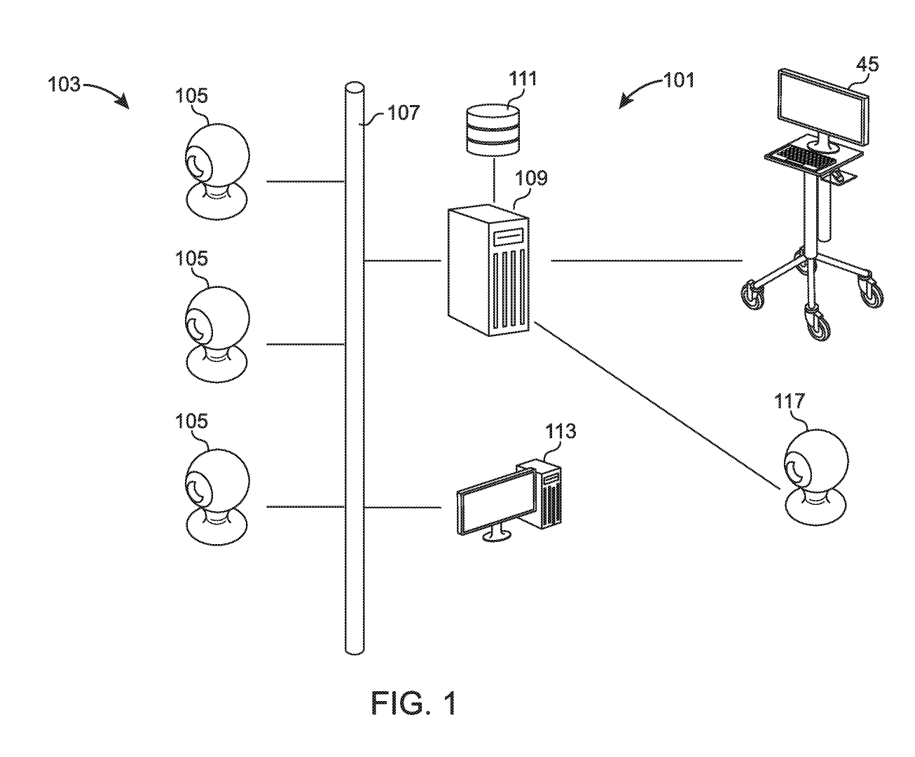

Still further, such systems may additionally or alternatively include, but are not necessarily limited to, the hydraulic bed sensor and system for non-invasive monitoring of physiological data described in U.S. Utility patent application Ser. No. 13/600,539, filed Aug. 31, 2012, the entire disclosure of which is incorporated herein by reference and/or may include, but are not necessarily limited to, the activity analysis, fall detection and risk assessment systems and methods described in U.S. Utility patent application Ser. Nos. 13/871,816, filed Apr. 26, 2013, and 14/169,508, filed Jan. 31, 2014, the entire disclosures of which is incorporated herein by reference. In particular, the '308 application describes the use of depth image data from at least one depth camera associated with a particular patient, and generating at least one three-dimensional object based on the depth image data. See, e.g., FIGS. 1-5, and paragraphs 22-57, describing same. As contemplated herein, any or all of the above systems and methods of these other applications may be used to perform the gait analysis contemplated herein.

Throughout this disclosure, the term “computer” describes hardware which generally implements functionality provided by digital computing technology, particularly computing functionality associated with microprocessors. The term “computer” is not intended to be limited to any specific type of computing device, but it is intended to be inclusive of all computational devices including, but not limited to: processing devices, microprocessors, personal computers, desktop computers, laptop computers, workstations, terminals, servers, clients, portable computers, handheld computers, smart, phones, tablet computers, mobile devices, server farms, hardware appliances, minicomputers, mainframe computers, video game consoles, handheld video game products, and wearable computing devices including but not limited to eyewear, wrist-wear, pendants, and clip-on devices.

As used herein, a “computer” is necessarily an abstraction of the functionality provided by a single computer device outfitted with the hardware and accessories typical of computers in a particular role. By way of example and not limitation, the term “computer” in reference to a laptop computer would be understood by one of ordinary skill in the art to include the functionality provided by pointer-based input devices, such as a mouse or track pad, whereas the terra “computer” used in reference to an enterprise-class server would be understood by one of ordinary skill in the art to include the functionality provided by redundant systems, such as RAID drives and dual power supplies.

It is also well known to those of ordinary skill in the art that the functionality of a single computer may be distributed across a number of individual machines. This distribution may be functional, as where specific machines perform specific tasks; or, balanced, as where each machine is capable of performing most or all functions of any other machine and is assigned tasks based on its available resources at a point in time. Thus, the term “computer” as used herein, can refer to a single, standalone, self-contained device or to a plurality of machines working together or independently, including without limitation: a network server farm, “cloud” computing system, software-as-a-service, or other distributed, or collaborative computer networks.

Those of ordinary skill in the art also appreciate that some devices which are not conventionally thought of as “computers” nevertheless exhibit the characteristics of a “computer” in certain contexts. Where such a device is performing the functions of a “computer” as described herein, the term “computer” includes such devices to that extent. Devices of this type include but are not limited to: network hardware, print servers, file servers, NAS and SAN, load balancers, and any other hardware capable of interacting with the systems and methods described herein in the matter of a conventional “computer.”

Throughout this disclosure, the term “software” refers to code objects, program, logic, command structures, data structures and definitions, source code, executable and/or binary files, machine code, object code, compiled libraries, implementations, algorithms, libraries, or any instruction or set of instructions capable of being executed, by a computer processor, or capable of being converted into a form capable of being executed by a computer processor, including without limitation virtual processors, or by the use of run-time environments, virtual machines, and/or interpreters. Those of ordinary skill in the art recognize that software can be wired or embedded into hardware, including without limitation onto a microchip, and still be considered “software” within the meaning of this disclosure. For purposes of this disclosure, soft ware includes without limitation: instructions stored or storable in RAM, ROM, flash memory BIOS, CMOS, mother and daughter board circuitry, hardware controllers, USB controllers or hosts, peripheral, devices and controllers, video cards, audio controllers, network cards, Bluetooth® and other wireless communication devices, virtual, memory, storage devices and associated controllers, firmware, and device drivers. The systems and methods described here are contemplated to use computers and computer software typically stored in a computer- or machine-readable storage medium or memory.

Throughout this disclosure, terms used herein to describe or reference media holding software, including without limitation terms such as “media,” “storage media,” and “memory,” may include or exclude transitory media such as signals and carrier waves.

Throughout this-disclosure, the term “network” generally refers to a voice, data, or other telecommunications network over which computers communicate with each other. The term “server” generally refers to a computer providing a service over a network, and a “client” generally refers to a computer accessing or using a service provided by a server over a network. Those having ordinary skill in the art will appreciate that the terms “server” and “client” may refer to hardware, software, and/or a combination of hardware and software, depending on context. Those having ordinary skill in the art will further appreciate that the terms “server” and “client” may refer to endpoints of a network communication or network connection, including but not necessarily limited to a network socket connection. Those having ordinary skill in the art will further appreciate that a “server” may comprise a plurality of software and/or hardware servers delivering a service or set of services. Those having ordinary skill in the art will further appreciate that the term “host” may, in noun form, refer to an endpoint of a network communication or network (e.g., “a remote host”), or may, in verb form, refer to a server providing a service over a network (“hosts a website”), or an access point for a service over a network.

Throughout this disclosure, the term “real time” refers to software operating within operational deadlines for a given event to commence or complete, or for a given module, software, or system to respond, and generally invokes that the response or performance time is, in ordinary user perception and considered the technological context, effectively generally cotemporaneous with a reference event. Those of ordinary skill in the art understand that “real time” does not literally mean the system processes input and/or responds instantaneously, but rather that the system processes and/or responds rapidly enough that the processing or response time is within the general human perception of the passage of real time in the operational contest of the program. Those of ordinary skill in the art understand that, where the operational context is a graphical user interface, “real time” normally implies a response time of no more than one second of actual time, with milliseconds or microseconds being preferable. However, those of ordinary skill in the art also understand that, under other operational contexts, a system operating in “real time” may exhibit delays longer than one second, particularly where network operations are involved.

The systems and methods described herein utilize the analysis of an individual's gait when entering a care facility, or when interacting with elements of a care facility, to determine if the individual has an increased likelihood of an urgent care condition in real time or near real time. To do this, the system generally looks at gait to determine to specific pieces, of information and is looking for elements of gait which either 1) indicate that the individual's gait shows an element corresponding with a population that is known to have had an urgent health condition or 2) indicate that the individual's gait corresponds to an individual that has additional stored health information which is accessible to the system.

In discussing gait, the systems and methods may utilize two different forms of analysis. The first is the walking gait of an individual who arrives at the facility under their own power. This gait will generally be detected either within the facility's admission area, or even outside it (such as in a parking lot). The second gait relates to the ability to the person to initiate walking. this can be used if a person arrives at the facility, or later is in a position, where they are seated and standing or leaving a bed. Both these types of systems are discussed in depth in the above documents that are incorporated herein.

The gait is then analyzed in two general fashions. The first is to look for indicators within the gait of a particular emergent situation which can be identified by elements of the gait which exist across multiple individuals having the same condition. That is, if specifics of the person's gait is indicative of someone who has suffered or is soon to suffer an urgent medical event. In many respects, this will often be determination of the likelihood of a fall and can also relate to conditions such as a cardiac, event or a stroke which could be cause of the fall.

The second method is to attempt to identify the individual based on specifics of their gait. From this, medical records and prior information may be quickly and securely retrieved from them regardless of the facility they are at or if they have ever visited the facility or any related facilities previously. This information can indicate a higher probability of a specific event leading to their desired admission or to obtain additional information which may be valuable in proscribing a treatment. Further, it also allows for their specific prior gait data to be compared against their current gait to locate additional information in the form of deviations from the prior recorded values.

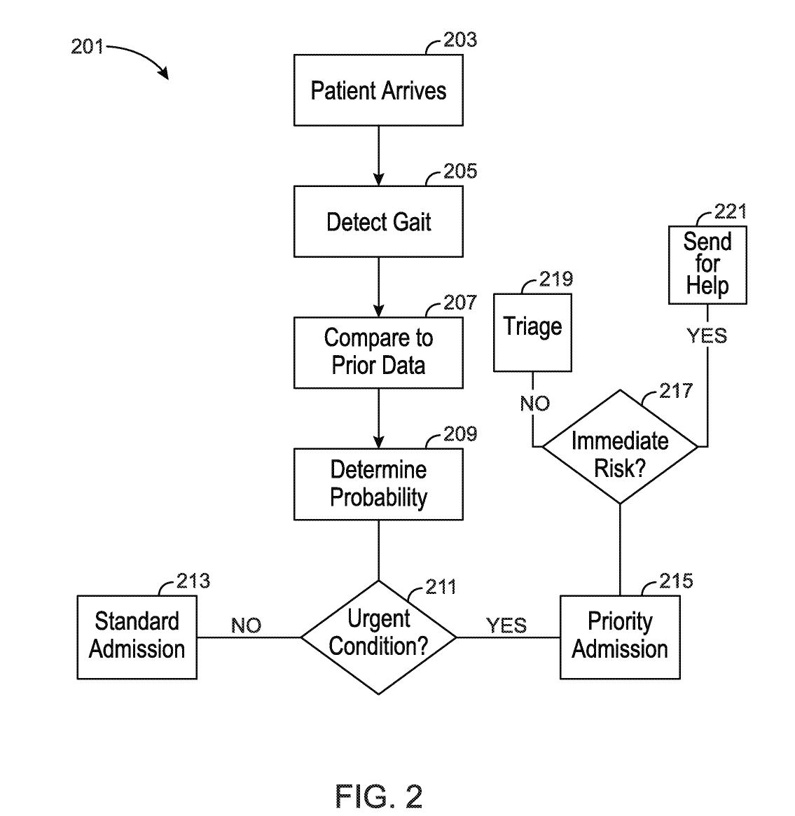

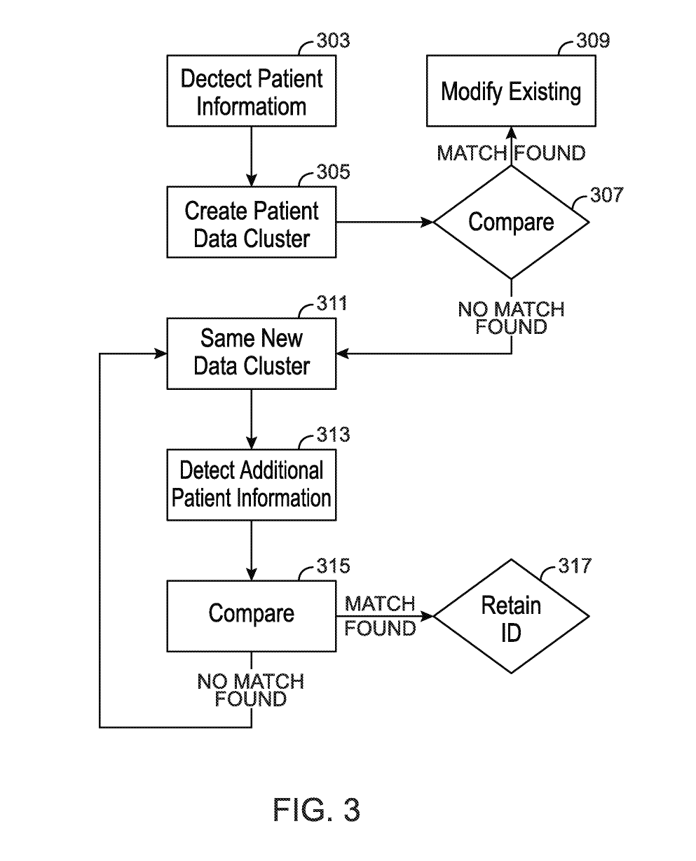

The following description should be understood with respect to the FIGS, which provide an embodiment of the present systems and methods. FIG. 1 depicts an exemplary system implementing a system for prioritizing admissions of a patient based upon an analysis of the patient's gait FIG. 2 depicts an exemplary embodiment of a method for prioritizing admissions of a patient based upon an analysis of the patient's gait related to detection of an emergent condition and FIG. 3 depicts an exemplary embodiment of a method for retrieving past medical history based on a patient's gait. The methods depicted in FIGS. 2 and 3 may be implemented using the systems depicted in FIG. 1, and the system depicted in FIG. 1 may implement the method depicted in FIGS. 2 and 3.

Referring now to FIG. 1, hardware systems, and software systems, which may be used in an embodiment to implement the present invention are depicted. The depicted system of FIG. 1 includes a server (109) with an attached storage media (111) connected via a network (107) to a sensing (103) or imaging (103) system. The server (109) is also connected via the network (107) to a triage workstation (113). The depicted sewer (109) may be any type of computer server known in the art, including, without limitation, cloud computing services and systems. Similarly, the data storage (111) may be any type of data storage known in the art. Typically, data storage (111) will comprise a structured database system, which may be a relational database system, such as an SQL database, or any other commercial or open source database system known or used in the art. The database (111) may be stored on an internal component of the server (109), such as a hard drive or RAID drive, or may be communicably connected to the server, and reside on a separate or independent system. The particular architecture in an embodiment will depend upon, among other things, the budget and scale of operations of the hospital in which the systems are deployed.

The network (107) is depicted schematically as a LAN, but one of ordinary skill in the art will understand that a wireless network may be substituted for the LAN as a functional equivalent. That is, the devices depicted in FIG. 1 as communicating via the LAN (107) may also communicate with one another wirelessly using any number of wireless network communication protocols, including, without limitation, the 802.11 family of protocols.

The triage workstation (113) may be a general or special purpose computer, but is more commonly a general purpose computer containing software programmed to manage hospital admissions. The triage computer (113) is typically deployed in an urgent or emergent care center, near the waiting area, and is used by the admitting nurse or staff to input data provided by entering patients, concerning their medical condition, demographic information, medical history, identity, and other information which may be useful in diagnosing and treating the condition that motivated the patient to come to the emergency room in the first place.

The sensing system (103) may comprise any number of different types of sensors, and in the depicted embodiment comprises a plurality of cameras (105). Although a “webcam” style camera (105) is depicted, one of ordinary skill in the art will understand that this is merely a symbolic representation of imaging technology, which runs the gamut from ordinary webcams to sophisticated infrared cameras and depth sensors, including high-resolution cameras capable of forming a three-dimensional image of the subject. Although three cameras are depicted in FIG. 1, the system may comprise one or more imaging devices, which are in communication with the server (109). Further, the system (101) may also include workstations (115) to provide information directly to the server (109). These may be part of a secure network such as within a medical facility for entering of data during a patient visit. These workstations (115) may al so include cameras (117) for collecting image data, and specifically data related to gait, during non-emergent care visits.

Referring now to FIG. 2, the system of FIG. 1 generally is operated, to detect an urgent medical situation anonymously. It is important to recognize that the term “anonymous” is used here not to indicate that the patient is unknown or unknowable, but that the patient has not yet been identified as a specific person with a known medical history, even if the person has actually been identified in a traditional fashion (e.g. their passport has been reviewed). In effect, the individual upon entering the facility is anonymous because they have not yet been identified as a particular individual with known health characteristics.

Generally, the method (201) begins when a patient arrives (203) at a healthcare facility, due to an urgent or emergent healthcare condition whether real or perceived. As an example, the patient may fee having chest pains. These may be caused by coronary artery disease which is a blockage in the heart blood vessels that reduces blood flow and oxygen to the heart. If sufficiently severe it is indicative of an urgent medical threat such as a heart attack. A second option is that this is caused by complications of pneumonia which, while a medical condition requiring treatment, generally does not require urgent attention. It could also be indicative of acid reflux, a temporary condition that generally requires only minor medical attention.

Currently, when such a patient arrives (203) at the admission room of the care facility (except when arriving by ambulance where their arrival may be expected), they are quickly seen by a triage nurse who may ask a few questions to determine if the patient has an urgent condition. For example, an indication of active chest pain is concerning as it may indicate a heart condition requiring rapid response, however, as the patient is currently stable, it will often result in them getting a classification of ESI-2 (needs attention, but not expected to deteriorate) in accordance with the ESI triage standard. Further, a nurse may ask about the types of pain in an attempt to determine which of the above (or any of a variety of other possible conditions) may be present or may attempt to obtain additional readily available health information such as body temperature, blood oxidation rate, pulse rate, breathing rate, etc.

The problem with this methodology of admissions is multiple. In the first instance, the descriptions of pain, and the understanding of the one hearing them, are both subjective and subject to multiple points of communication error. The second is that it requires the individual to be able to communicate with the triage nurse. This may not be the case if there is a language barrier, for instance. A third problem is that the triage nurse needs to be able to remember how to classify a patient's answers (and the questions to ask) to make sure that the information obtained is valuable. A third is that even simple examinations, such as obtaining pulse rate take time and take the attention of the nurse on this patient.

In the present systems and methods, the triage nurse's ability to gather information is generally still used, but it is generally supplanted by initial computer evaluation of the patient to give the nurse increased information and particularly to direct the nurse to potentially more urgent cases initially. The computer may, in alternative embodiments or certain situations, bypass the nurse and initiate an action on its own. However, this is generally not preferred as professional judgement is still seen as valuable and the information provided by the system is usually intended to upgrade a patient to a higher level of urgency, not to actually assign a level of urgency, as the information the systems and methods discussed herein can provide are often insufficient to make a medical diagnosis. Therefore, the systems and methods are focused on providing additional information to the decision maker quicker. This process occurs as follows.

As discussed above, an imaging system (103) is deployed in the entrance area of the emergent care center, and is positioned so as to fee able to capture image data for generally all persons entering the emergency care center. This process is performed automatically for all persons who enter the area, whether healthy or not. Thus, should one individual arrive with another, both will be imaged and searched. The sensing system (103) detects the gait (205) of the persons entering the emergency care center and communicates data concerning the detected gait of each person to the server (109). The server (109) contains software programming configured to compare (207) the received data from, the sensing system (103) to an existing database of gait data. This database of gait data will typically be stored in data storage (111). This database comprises gait data gathered from prior visitors to the emergency room, as well as data concerning any conditions that such patients were found to have, or reported to have, and the ultimate disposition of their emergency previously. By way of example and not limitation, the database may include data for a patient who entered exhibiting a particular gait trait, and subsequently suffered a cardiac event.

This comparison process (207) uses datamining techniques known in the art to identify a subset of patients in the gait data having a gait profile most similar to that observed by the sensing system and is generally looking for specific traits of gait which are shared across individuals who are found to have similar health issues (209). For example, in the event of a stroke, individuals will often have difficulty effectively using a portion of their body due to weakness. This can manifest as a similar gait difference across multiple individuals regardless of individual traits of their gait as a difference in the gait showing them favoring one side over the other or of having a specific difficulty in walking.

Corresponding health conditions for patients in such subset of the gait data are then, identified and a probability is determined of the likelihood that the subject of the received gait data from the sensing system (103) may have one of the corresponding health conditions (209). The server (109) then determines whether the high-probability conditions, if any, are emergent or urgent conditions (211). If not (213), the patient may be triaged and/or admitted as normal. If any of the high-probability conditions are determined to be emergent or urgent conditions, the patient is given priority admission (215). Priority admission (215) may relate to how immediate the detected concern is (217). If extremely urgent, request for emergency personnel, maybe transmitted automatically (221). Alternatively, it may mean that the patient is triaged in a priority fashion by the triage nurse (219) or provided with other assistance which may reduce the likelihood that the situation escalates.

A good example of how the systems and methods can be used to improve response and triage is as follows. As discussed in many of the applications incorporated above, the system may detect that the individual, based on their gait, has a high fall risk. Should this determination be made, the system will often indicate to the triage nurse that this patient needs to be quickly provided with a wheelchair or immediately given a seat upon their entry to the facility. This provides for immediate care (221). Further, the increased fall risk probability is valuable piece of information which could indicate a urgent concern. As such, even if the patient does not actually fall and the intervention is not actually available the patient may be indicated to the triage nurse as one who needs to be triaged before others in the waiting area (regardless of position in line) (219).

To continue the example, the patient at an increased fall risk arrives and is quickly detected. However, before a nurse can respond, the patient is quickly helped to a seat by a second, individual who arrived with the patient. The second individual then comes to the triage nurse station and is waiting in line to have the patient initially evaluated, and triaged. The fall risk indication gives the triage nurse a potentially critical piece of information they did not otherwise have, particularly if they would have been unable to see the patient as they arrived.

Increased fall risk corresponds to many symptoms which can correspond to ESI level 1 and 2 conditions. As an example weaknesses and dizziness present increased fall risk and, if combined with a low heart rate, is an ESI level 1 indicator. Without the determination of the fall risk being provided by the system, the triage nurse has no indication of any information about the patient and has no reason to triage them above anyone else in the waiting area using priority triage (219). With this piece of information, the triage nurse can immediately work with the second individual and ask questions or go and talk with the first one, or perform simple tests to get a better assessment and triage determination.

Additionally, the server (109) may determine whether the high-probability health condition poses an immediate risk to the patient (217). For example, the server may determine that, based on the gait data and the comparison (207) to the gait data in data storage (111), the patient is going to suffer an imminent cardiac event or stroke. In this case, the server may automatically send for additional assistance (221), such as by requesting a gurney or bed and the arrival of specialized medical personnel so that the patient can be seen immediately and the event prevented or immediately addressed. However, even if additional resources are not requested, the patient may still be triaged (219) as a priority admission (215) (e.g. EIS-1 or 2) based on nothing other than the fall risk.

It should be recognized that gait analysis can also be used in conjunction with the action of standing or sitting. For example, if a patient is determined to be a fall risk, they may be immediately supplied with a chair or gurney, or may have sat or laid down on one when they entered. Such a gurney or chair, (and even chairs or benches in the admissions area itself) may be equipped with bed sensors and other sensing devices and this may be used to detect that the person that was identified as a fall risk because of an imminent or-occurring cardiac event or other emergent health condition. In such a situation, the system could actually immediately classify the individual as an ESI level 1 and automatically alert appropriate response teams (221). This can even be done before the individual has encountered the triage nurse.

A key element of the above analysis of the individual patient is that it is all performed anonymously. There is no need to have identified the patient at the time both this initial determination occurs and during the initial upgrading of the patient to the triage nurse. Instead of the nurse being focused on obtaining information which may be indicative of an urgent condition, the increased likelihood of an urgent condition can quickly be determined and the nurse can now focus on verifying that condition, disproving that condition, or better classifying the nature of the condition. Further, there is no need for the patient to have even interacted with any individual in the admissions area to start the care process. Instead, the computer system identified the patient as a fall risk and then simply notified the triage staff using the triage workstation (113) or other system. In effect, the systems and methods discussed herein have initially triaged patients into those more likely to need emergency care and those less likely, the system then has the triage nurse triage patients beginning with the most concerning first.

While anonymous identification is desirable due to the speed at which it can be performed, another element of admissions which can be performed is the rapid, potential identification of the patient. Specifically, the patient, or someone who arrives with the patient, may be identifiable from their gait. This can provide the triage nurse with additional information to request from the patient which may help more positively identify, or eliminate, the likelihood that the condition exists.

For example, if a person arrived in the emergency room and no emergent condition was identified from their gait, the systems and methods need not stop here. Once the system has either identified a potentially urgent situation based on specifics of the gait from comparison against the population generally, the system may continue to analyze the gait. In particular, in a second level of analysis, the system may analyze the gait to determine if the patient can be identified.

The algorithms used by the server to make these determinations are generally implemented in software stored on a computer readable medium within the server, as would be understood to one of ordinary skill in the art and the same system of FIG. 1 may be used in this determination. The software is executed by a microprocessor or set of microprocessors associated with the server (109). The software will implement one or more algorithms for conducting datamining operations. Such algorithms are known in the art, and may take into account, among other things, factors such as: patient weight, patient mass, patient walks, patient height, and gait data. All of this information may be generated automatically on the basis of observations made by the sensing system (103). This data may also, or alternatively, be refined, or supplemented by patient-specific data provided or entered by hospital staff, such as, but not limited to, using mobile computer systems (115) or recorded by camera (117). This, and other data, may be included in the gait data in data storage (111).

The sensing systems (103) and (117) may use, among other things, depth sensors and bed sensors described in the above-referenced patent applications, or other similar types of sensors. These systems may be used in an embodiment to identity a hospital patient and distinguish that patient from other people or objects in the field of evaluation. This identification is accomplished through the use of a depth sensor which produces a three-dimensional representation of the field of evaluation, and/or a bed sensor. These sensors may work together to create a patient specific data cluster. This data cluster comprises data about the patient based upon information collected by the depth sensor and/or bed sensor. These sensors detect information about the patient's movements, including gait, and over time develop a sufficient body of data to uniquely identify the patient on the basis of that data. That is, the data gathered by the imaging systems is provided to the server (109) and stored in data storage (111). This data cluster may also be referred to herein as a patient data profile. Subsequently, when the imaging systems detect the presence of a person, a second data cluster is generated, and compared to data clusters already existing in data storage (111). This comparison can determine whether the newly-detected data cluster is indicative of the same person as a previously detected data cluster, and return such an indication to application software to identify a patient. Using proprietary algorithms, the patient can be identified while preserving the patient's privacy, because the patient's name and identity need never be known.

For example, patient medical data and records may be associated with a patient data profile. This effectively allows the patient's data profile (their gait) to be used as an access key for the patient's medical records, similar to a motion-based fingerprint or retinal scan. However, unlike a fingerprint or retinal scan, the patient need not necessarily cooperate in providing the biometric input. Rather, the biometric input is gathered passively by the imaging systems, with or without the patient's active participation. This is particularly useful in emergency rooms where the determination can be made upon entry without any need to request cooperation. Such a system may also be useful in identifying a patient who is incapable of identifying him or herself often for the same reasons.

Referring to FIG. 3, the system of FIG. 1 is generally operated as follows in identifying the individual. The camera systems described with respect to FIG. 1 are used to detect patient Information (303). This information is then provided to the server system (109) where software executing on the server (109) processor creates (305) a patient data cluster based upon the information received from the imaging systems (103). This data cluster may be created in data storage (111) or in another memory of the server (109), such as program memory for an application, or local storage.

Next, the server (109), again via software executing on a processor of the server (109), compares (307) the newly created patient data cluster with the set of previously created patient data clusters in data storage (111). This comparison is done using any number of algorithms, as the data will generally not match exactly. Rather, trends and profiles and patterns are sought identified, and matched, based on algorithms. If a matching existing data set is found, this data set may be modified or updated (309) based upon the newly created patient data cluster. In this instance, the newly detected patient information has been matched to a previously detected patient, and the newly detected information may be used to modify or update the prior profile to further refine it or otherwise improve its completeness and accuracy. In this case, the newly created data cluster may be deleted or discarded, as it is duplicative. However, in an embodiment, as it is possible that the algorithms have misidentified a match. Later, manual checking by a human operator can be used to train the system by identifying incorrect matches. In such cases, the newly created data cluster will be stared as a newly identified patient, separate from the matched patient.

If no match is found, then the new data cluster is stored (311) in data storage (111) as a new patient. Again, it is important to understand that this data pertains only to perceptible information about the patient based upon external sensors, and not any demographic or identifying information pro vided by the patient or anybody else. That is, the patient is identified based entirely upon observable characteristics. However, these characteristics are generally subtle and nuanced, and not readily perceptible to the unaided human eye. Again, this process may involve a later step of human training by reviewing the newly created data cluster, and confirming that it does not correspond to a previously existing patient. If it does, the cluster may be merged into or otherwise used to modify the existing patient data (309) in order to further train the system. This technique is known in the art as supervised learning.

If the new data cluster is retained in data storage (311), then a new patient has been detected. Next, the sensors (105) detect (313) additional patient information, and the same type of comparison (315) is carried out. If this comparison (315) reveals that the newly detected patient information matches the previously stored patient information (311), then an identifier (317) associated with the newly stored data cluster (311) is returned to application software. If there is no match, then the process repeats at the store new data step (311) as previously described.

The unique user identifier (317) is generally a serial number or other unique identifier associated with the data cluster. This identifier, again, does not contain any personally identifiable information, but is merely a key or index into the data cluster, which is more human readable and understandable than the general mass of information contained in the patient data cluster. This ID may also be a foreign key into other databases, such as patient medical record databases. In this fashion, the biometric information gathered by the sensors (105) can be used to access patient medical records associated with the ID (317) without the user having to know the patient's identity.

This allows the medical records to be called up, viewed, and accessed by a medical professional or other caregiver without having to know who the patient is, and without compromising the patient's privacy. Further, because of the anonymity of the patient, the system may fee used to additionally or alternatively allow a patient to be identified without the patient being identified via any form of hospital identifier. This arrangement can allow for a third party system that is involved in monitoring and risk assessment to identify and provide notifications of patients, without the third party having to have access to any data on the hospital servers or network. This arrangement can provide for improved data security for the patient and the hospital since it can allow a third party to perform monitoring of particular patients while allowing identifying information, and protected health information, on the hospital's systems to be shielded from the third party. Further, gait information can be used to locate information on public servers or other health facilities without identifying the patient. This could allow records of a facility which is closed to actually be securely accessed.

The advantage of loading the medical record quickly (309), and particularly if a medical record can be loaded quickly which is not otherwise at the hospital because the patient's identity can be verified by their gait, the triage nurse can be provided with targeted questions or relevant information on why the patient may be present. For example, the system in the chest pain situation, may provide the nurse with specific questions to ask quickly such as the exact nature of the pain (dull, burning), or questions regarding immediately prior activities (how long has this been going on, what were you doing when it started). The triage nurse, in an embodiment, may even be provided with particular tests to have the patient perform such as if they can focus on an object. These questions may be based on quickly identifying or ruling out a likely issue,

For example, if the patient medical record is palled up (309) and it is seen that the patient recently had surgery in their chest, the first consideration is that the pain may be a surgical complication or infection, which requires quite a different medical response to a heart attack. Similarly, if the patient's record shows that they were recently prescribed a new medication which is known to cause acid reflux, that may be variable information. Similarly, if the patient is pregnant, that can be immediately identified and may influence the determination.

The key in collecting the admission information based on gait is twofold. In the first instance, the information is obtained automatically by the computer and is done so as the patient is entering the facility. In this way, the need to interact with the triage nurse as the first stage of triage is eliminated. Patents can be triaged (at least initially) automatically without any need for them to interact with any person in the admissions area. This means that even though the triage nurse likely will still be necessary to provide additional information or to clarify questions and make the triage decision, patients are already presorted based on an expected urgency before they have to talk with the triage nurse. Thus, should there be a line at the triage nurse, for example, patients may be given priority based on expected urgency of condition as opposed to how they arrive.

It should be recognized that once a patient has been identified by gait, the computer can still continue and may actually compare their current gait against their known gait looking for anomalies. As discussed above in conjunction with comparisons for people with known medical concerns having particular deviations in their gait, identifying a specific deviation in this specific patient can also provide an immediate indication of urgency and possible reason for being in the admissions area.

It is also important to note here that at the time these comparisons are occurring, the patient is still anonymous. They generally still have not been identified as a particular person, they are simply a person identified as having a particular gait. Should a medical record be located for a person matching the gait, this can be provided to the triage nurse highlighting relevant issues. Alternatively, to further protect confidentiality, a question many be provided to the triage nurse to attempt identification. For example, if the gait was determined to match an Individual by the name of “John Notajondoe” who was born Jan. 1, 1961 the triage nurse could be provided the first question to ask of the patient being their name. If the response back was “John Notajondoe” the system would load that particular medical record and provide it to the triage nurse without further questions being asked, to the triage nurse may be cued to ask for birthdate. Upon both those items matching the record, the record could be immediately provided to the triage nurse to provide additional information.

In the above situations, the patient's gait is being compared against other patient's previously admitted to the facility to determine if they are a prior patient and medical records may be available due to that prior admission. If this individual is not identified as a patient at the hospital, others who arrived with the patient may be similarly analyzed or the gait information maybe provided to other hospital databases to attempt to find a match. An advantage (c)fusing gait as an identifier in this case is that it is very anonymous and therefore can be used on virtually anyone without significant risk of confidentiality being lost. Specifically, gait analysis can generally not be used to identify an individual unless a database already has information on the gait, which is indicative of them already having medical records on them. Thus, the gait information can be used without as much concern of violations of HIPAA or other healthcare privacy acts.

For example, consider a situation the patient was an elderly man being brought in by a younger woman. The man's gait did not register with the system, but the woman's did and she was identified as “Jane Notajondoe”. Further, from Jane's medical, record it is known her father's name is “John Notajondoe” and be has been treated at the hospital previously for cardiac symptoms but his gait is unavailable, the system may quickly cue the triage nurse to ask the patient's name. If the patient identifies himself (or is otherwise identified) as “John Notajondoe”, John's record may immediately be loaded and the triage nurse's question may focus on possible cardiac concerns. Note that while Jane was the hospital patient and her record was used in this identification, she is actually never identified to the nurse and her medical record is not accessible, her anonymity is thus preserved.

While the above has focused on the concept of intelligent admissions, the system need not be only used at admissions. Once a patient, has been admitted and treated, or is in the process of being treated, the patient's condition may be continually assessed automatically using similar systems and methods. That is, sensors (117) may be deployed within the hospital treatment areas, such as within the patient's room, and continually monitor the patient's gait to determine whether the patient is improving, or is in condition for discharge. This monitoring may be performed when the patient walks to the restroom, or otherwise gets out of bed. Additionally, this evaluation may be modified, refined, or otherwise revised, by taking into account additional medical information about the patient provided by treating healthcare staff. For example, nurses and doctors may observe the patient and note in the patient's medical records prior conditions or family histories of certain conditions, which may assist in further refining the prediction of an urgent condition or immediate risk to the patient. This data is typically entered by nurses or doctors using mobile computer systems (115) in the treatment areas of the hospital.

In an embodiment, this data can also be used to predict the discharge rate for a patient based on, among other things, an evaluation of the patient's gait and a comparison of the gait to prior data (111). By way of example, and not limitation, trends may be identified in prior data showing that patients exhibiting a pattern of gait change similar to that of a given patient undergoing treatment, and that patients exhibiting such pattern are typically discharged within a certain amount of time. This amount of time may be a discrete amount (e.g., two days) or a range of time (four to five days). This data may be useful to hospital staff in estimating when, a patient is likely to be ready for discharge, which data may be used to assist in making the discharge decision. For example, where a patient appears well, and is ready to leave, but the gait analysis suggests that the patient should remain hospitalized for two more days, treating staff may use this information to conduct further testing or assessment of the patient before discharging. Conversely, where a patient insists upon remaining, gait data may be used to determine that, based on the patient's gait, the patient is at or beyond the typical point at which discharge is appropriate. This data may be helpful to hospital staff in determining whether a given patient is an “eggshell” patient or has a genuine medical condition that merits further investigation.

While the invention has been disclosed in conjunction with a description of certain embodiments, including those that are currently believed to be the preferred embodiments, the detailed description is intended to be illustrative and should not be understood to limit the scope of the present disclosure. As would be understood by one of ordinary skill in the art, embodiments other than those described in detail herein are encompassed by the present invention. Modifications and variations of the described embodiments may be made without departing from the spirit and scope of the invention.

1. A system for assisting in determining the urgency of admission in an admitting room of a care facility, the system comprising:
a plurality of depth cameras, said plurality imaging an individual walking into said admitting room to determine said individual's gait;
a computer, said computer taking in said imaging and comparing said individual's gait against stored gaits;
if said individual's gait matches a specific gait of a prior patient at said care facility, prioritizing admission of said individual based on an expected priority of said prior patient; and
if said individual's gate does not match stored gaits, providing said individual normal admission to said care facility.
